(19) United States
(12) Patent Application Publication
(10) Pub. No.: US 2007/0276186 A1
(43) Pub. Date: Nov. 29, 2007

(54) Endoscope, In Particular For The Intubation Of A Respiratory Tract

(76) Inventor: Martin Renner, Liptingen (DE)

Correspondence Address: ST. ONGE STEWARD JOHNSTON & REENS, LLC, 986 BEDFORD STREET, STAMFORD, CT, 06905-5619, US

(21) Appl. No.: 11/752,486

(22) Filed: May 23, 2007

(30) Foreign Application Priority Data
May 24, 2006 (DE) 10 2006 025 621.2

(51) Int. Cl. A61B 1/06; A61B 1/00

(52) U.S. Cl. 600/164; 600/120

(57) ABSTRACT

An endoscope for the intubation of a respiratory tract, comprises a shaft and an eyepiece at the proximal end of an endoscope head and comprises a joint between the shaft and the eyepiece for angling the eyepiece away in relation to a longitudinal direction of the shaft. The joint is formed in such a way that the eyepiece can be angled away in a first plane. In addition, the joint is formed in such a way that the eyepiece can be angled away at least in a second plane, which runs transversely in relation to the first plane.

### CROSS REFERENCE TO FOREIGN APPLICATION

The present application claims priority of German patent application No. 10 2006 025 621.2 filed on May 24, 2006.

### BACKGROUND OF THE INVENTION

The invention generally relates to endoscopes. The invention specifically relates to an endoscope for the intubation of a respiratory tract which can be used in anesthesia and emergency medicine.

An instrument of this type is known for example from the German company brochure of Karl Storz GmbH & Co. KG, Tuttlingen, “Karl Storz-Endoskope, Endoskope for Anasthesie und Notfallmedizin” [Karl Storz endoscopes, endoscopes for Anästesie and emergency medicine], 3rd edition January 2004, page AN-SET 9 B. An endoscope provided with the instrument number 10330 B is known from this brochure.

The known endoscope is used for the intubation of the respiratory tracts of a human, the distal end of the endoscope being inserted through the nose or the mouth of a patient into the trachea for the optical monitoring of the insertion of an intubation tube. The endoscope has an endoscope shaft and an endoscope head, an eyepiece being arranged at the proximal end of the endoscope head. The eyepiece and the shaft are connected to each other by means of a joint.

For the purposes of the present invention, a joint has at least two elements, which act on each other by rotation and/or translation.

The endoscope head of the known endoscope has a two-part housing, the joint being arranged between the two housing parts and the proximal end of the shaft running in the same longitudinal direction as the distal end of the endoscope head. The longitudinal direction of the endoscope shaft refers hereafter to that direction that is defined by the longitudinal direction of the proximal shaft end. The eyepiece can be angled away from a position of zero degrees, which may for example coincide with the longitudinal direction of the shaft.

At the distal end of the endoscope head, on the distal side of the joint, illuminating light is coupled via a connection into a light guiding system, which extends tip to the distal end of the shaft. Observation light of the imaging system is guided from the distal end of the shaft via an image carrying system through the joint to the eyepiece. The image carrying system has ordered light guiding fibres and/or a system of lenses for guiding and focusing the light. The image carrying system may be formed as a semiflexible or flexible light guide.

Such endoscopes arc used with preference in emergency medicine. A doctor bending over a patient holds the endoscope at its endoscope head, the distal end of the shaft being inserted into the patient's trachea. One of the doctor's hands is at the proximal end of the endoscope head and the other hand is at the distal end of the endoscope head, in order to be able to angle the eyepiece away in relation to the longitudinal direction of the shaft. The eyepiece, formed in such a way that it can be angled away, enables the doctor to assume a position in relation to the patient such that he does not have to be directly over the patient's nose and mouth orifices during the intervention.

However, the eyepiece of the endoscope that is known from the prior art can only be angled away in a defined plane in relation to the longitudinal direction of the shaft, with the result that the doctor's freedom of movement is restricted. In a situation in which the position of the doctor in relation to the patient is such that the doctor is at least partially over the patient, the doctor must remain in a position that is physically strenuous and awkward for him during the entire intervention. If no acceptable alignment of the eyepiece in relation to the shaft can be found for the doctor, the endoscope must either be reinserted into the trachea or the patient must be turned into a favourable position. This involves the risk of the patient suffering additional injuries. Turning of the endoscope about the longitudinal direction of the shaft is not possible in the case of a semiflexible shaft, or even a rigid shaft with a curved shape, when the endoscope is inserted in the trachea.

Furthermore, an endoscope with a flexible bending portion is known from DE 103 51 185 A1, the endoscope having between the distal end of the shaft and the proximal end of the endoscope head a working channel, for leading through a light guiding system, and instrument channels. The eyepiece, which is located at the proximal end of the endoscope head, can be bent away from its straight position by means of the bending portion. The bending portion is formed as a one-part tube with a spiral reinforcement along its longitudinal extent between a fixed endpiece at the proximal end of the endoscope head and a housing of the endoscope head. A bending portion has the disadvantage in comparison with a joint that it can become fatigued after being bent many times in different directions, i.e. it may become slack after the endoscope has been in use for some time. After the angling away of the eyepiece has undergone repeated changes in direction, the bending portion may have uneven, sinuously bent regions, because no fixed bending point is defined. Such bent regions of the bending portion through which the image carrying system for the observation passes lead to a corresponding sinuous shape of the light guiding system, which can impair the imaging quality of the image carrying system.

### SUMMARY OF THE INVENTION

The invention is based on the object of providing an endoscope of the type mentioned at the beginning which allows the doctor a greater freedom of movement during observation through the endoscope.

According to the invention, an endoscope for the intubation of a respiratory tract, is provided, comprising a shaft defining a longitudinal direction and having a proximal end, an endoscope head arranged at said proximal end of the shaft and carrying an eyepiece, a joint arranged on said endoscope head between the shaft and the eyepiece for angling the eyepiece away in relation to the longitudinal direction of the shaft, the joint being formed in such a way that the eyepiece can be angled away in a first plane and at least in a second plane which runs transversely in relation to the first plane.

Accordingly, the endoscope according to the invention not only allows the angling away of the eyepiece in a defined plane in relation to the longitudinal direction of the shaft. Rather, the eyepiece is formed in such a way that it can be angled away spatially in relation to the longitudinal direction of the shaft. The endoscope according to the invention advantageously offers the doctor increased freedom of movement during the intervention, since the relative alignability of the eyepiece in relation to the shaft is increased by at least a second angular range, which is provided by a second plane, transverse to the first plane. Moreover, the doctor can work without becoming fatigued, since he can remain in a physically relaxed position during the intervention.

A further advantage of the endoscope according to the invention is that it is easy to handle during the intervention, so that even doctors without many years of professional experience in the handling of endoscopes can use the endoscope of the present invention. On account of the spatial angling-away capability of the endoscope, it is not necessary during the insertion of the endoscope to pay attention to how the angled-away eyepiece is arranged in relation to the longitudinal direction of the shaft to enable the doctor to adopt a favourable position in relation to the patient. Even after insertion of the endoscope, the doctor can assume the position that is most suitable for him in relation to the patient, by angling the eyepiece away in the desired direction.

In addition, the present endoscope, which can be used in particular in the area of emergency medicine, permits time-saving application during the intervention. If the endoscope has already been inserted into the patient's trachea and the position of the patient in relation to the doctor is not suitable for further treatment, the eyepiece can be angled away in relation to the shaft into the most favourable spatial position, without having to reinsert the endoscope into the trachea.

In a preferred configuration of the invention, the joint of the endoscope is formed in such a way that the eyepiece can be angled away, at least in one of the first or second planes, up to an angle of approximately 50°, preferably to an angle of approximately 40° and more preferably to an angle of approximately 30°, in relation to the longitudinal direction of the shaft.

This measure has the advantage that the maximum angular range of one of the two planes, which is provided by the angling away of the eyepiece to both sides in relation to the longitudinal direction of the shaft, is 100° , 80° or 60°, and gives the doctor great freedom of movement.

In a further preferred configuration, the joint is formed as a ball joint.

This measure has the advantage that the joint can be produced in a structurally simple and low-cost way. Moreover, the ball joint allows the endoscope to be handled in such a way that it is easy to operate, since the desired angling-away position of the eyepiece in relation to the longitudinal direction of the shaft can be achieved in a direct way. The doctor does not have to consider during the intubation the angular increments in which the eyepiece must be angled away.

In a further preferred configuration, the ball joint has a two-part housing, a first housing part being connected to the shaft and a second housing part being connected to the eyepiece.

This measure has the advantage that the housing offers stable, compact protection for the light guiding system. Moreover, the endoscope head is formed in a structurally simple way, making it easy for the doctor to handle and grip it. The dividing of the housing into two has the effect at the same time of realizing the spatial angling away function in relation to the longitudinal direction of the shaft.

In a further preferred configuration, the first housing part is accommodated in the second housing part in a sliding manner with frictional engagement.

This measure has the advantage that the two housing parts are movable in relation to each other without any further technical expenditure being required. The frictional engagement creates a self-locking effect of the joint, with the result that the eyepiece maintains its spatial angled-away setting even when the doctor lets go of the eyepiece.

In a further preferred configuration, the first housing part has a spherical outer surface and the second housing part has a spherical inner surface, in which the first housing part is accommodated.

This measure has the advantage that the surface of the two housing parts is smooth and formed without projections so that the movement of the two housing parts in relation to each other is not restricted. Since both the outer surface of the first housing part and the inner surface of the second housing part are spherically formed, the two housing parts are connected to each other with an exact fit. Moreover, the spherical configuration of the two housing parts allows the angling away of the eyepiece in relation to the longitudinal direction of the shaft in more than one second plane, transverse to the first plane. This allows the eyepiece to be moved on a ball socket.

In a further preferred configuration, the first housing part is arranged in the second housing part in such a way that the second housing part extends beyond the maximum outside diameter of the spherical outer surface of the first housing part.

This measure has the advantage that the two housing parts are fastened to each other without any further technical measures.

In a further preferred configuration, the spherical outer surface of the first housing part has a radial constriction on the side facing away from the second housing part.

This measure has the advantage that the radial constriction extends the range of movement of the second housing part with respect to the first housing part, since the second housing part can be moved further over the outer surface of the first housing part and does not butt against it.

In a further preferred configuration, the radial constriction tapers, seen in the longitudinal direction away from the joint.

This measure has the advantage that the constriction, becoming radially smaller when seen in the longitudinal direction of the shaft, increases the maximum range of movement of the second housing part with respect to the first housing part.

In a further preferred configuration, the two housing parts act together as a stop to limit the angling away of the eyepiece.

This measure has the advantage that the maximum deflecting angle of the eyepiece in relation to the longitudinal direction of the shaft is restricted and the image carrying system that is led through the joint cannot be interrupted by excessive angling away.

In a preferred configuration that is an alternative to the ball joint, the joint is formed as a cardanic joint.

Also when in the form of a cardanic joint, the joint can be produced in a technically simple and low-cost way, to create a spatial angling away of the eyepiece.

In a further preferred configuration, the cardanic joint has a two-part housing and two crossed axes, a first housing part being connected to the shaft and a second housing part being connected to the eyepiece, and the crossed axes being arranged between the two housing parts.

This measure has the advantage that the housing offers compact and stable protection for the image carrying system. Moreover, the joint housing allows the doctor to be able to experience good handling of the endoscope during the intervention. The crossed axes between the two housing parts permit the angling away of the eyepiece with respect to the shaft in at least two planes arranged transversely in relation to each other.

In an alternative preferred configuration, the joint has at least two spaced-apart 1-axis joints.

This measure represents a further low-cost configuration of the joint that can be realized in a technically simple way, to permit spatial angling away of the eyepiece in relation to the longitudinal direction of the shaft.

In a further preferred configuration, the joint has a passage for leading through the image carrying system, the passage being free from projections.

This measure has the advantage that the image carrying system is protected from being damaged by the inner side of the joint when the eyepiece is angled away.

In a further preferred configuration, the joint has a protective tube for the image carrying system, the protective tube extending through the passage of the joint and accommodating the image carrying system.

This measure has the advantage that the sensitive image carrying system is protected by the protective tube even better from being damaged.

In a further preferred configuration, the protective tube extends around the image carrying system over its full circumference.

This measure has the advantage that the image carrying system is evenly protected from being damaged on all sides equally.

Further advantages and features emerge from the following description and the accompanying drawings.

It goes without saying that the features mentioned above and those still to be explained below can be used not only in the combination respectively specified but also in other combinations or on their own without departing from the scope of the present invention.

### BRIEF DESCRIPTION OF THE DRAWINGS

Exemplary embodiments of the invention are explained in more detail in the description which follows and are represented in the drawings, in which:

FIG. 1 shows a perspective view of an endoscope comprising an endoscope head and comprising an only partially represented shaft;

FIGS. 2A, 2B, 2C respectively show a side view of the endoscope in FIG. 1, FIG. 2A showing the endoscope with the eyepiece not angled away and FIGS. 2B and 2C showing the endoscope with the eyepiece in various angled-away positions;

FIG. 3 shows a cross section of the endoscope head in FIG. 1 in a not angled-away position;

FIGS. 4A, 4B respectively show a cross section of the endoscope head in FIG. 1 in the region of the joint in an angled-away position (FIG. 4A) and in a not angled-away position (FIG. 4B);

FIGS. 5A, 5B show a side view of an endoscope head (FIG. 5A) according to a further exemplary embodiment and a perspective exploded drawing of the joint (FIG. 5B); and

FIGS. 6A, 6B show a side view of the endoscope head (FIG. 6A) according to yet another exemplary embodiment and a perspective exploded drawing of the joint (FIG. 6B).

### DETAILED DESCRIPTION OF THE PREFERRED EXEMPLARY EMBODIMENTS

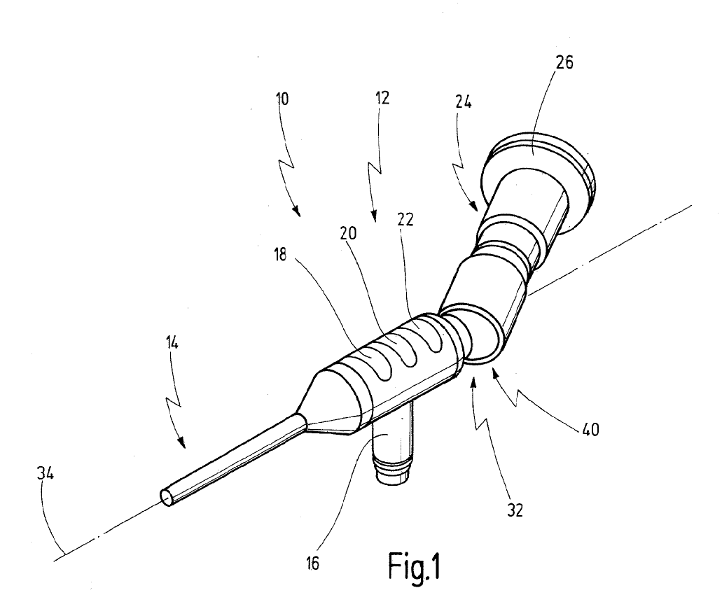

In FIGS. 1 to 3, an endoscope for the intubation of a respiratory tract is represented, provided with the general reference numeral 10.

The endoscope 10 has at its proximal end an endoscope head 12 and at its distal end a semiflexible or rigid endoscope shaft 14. The distal end of the endoscope head 12 has a connection 16 for a light guiding cable.

In addition, the distal end of the endoscope head 12 is formed with indents 18-22 in such a way that the doctor can place the tips of his fingers in these indents 18-22 while he is holding the endoscope 10. The proximal end of the endoscope head 12 has a focusing ring 24 and an eyepiece cup 26, which are arranged at the extreme proximal end of the endoscope head 12.

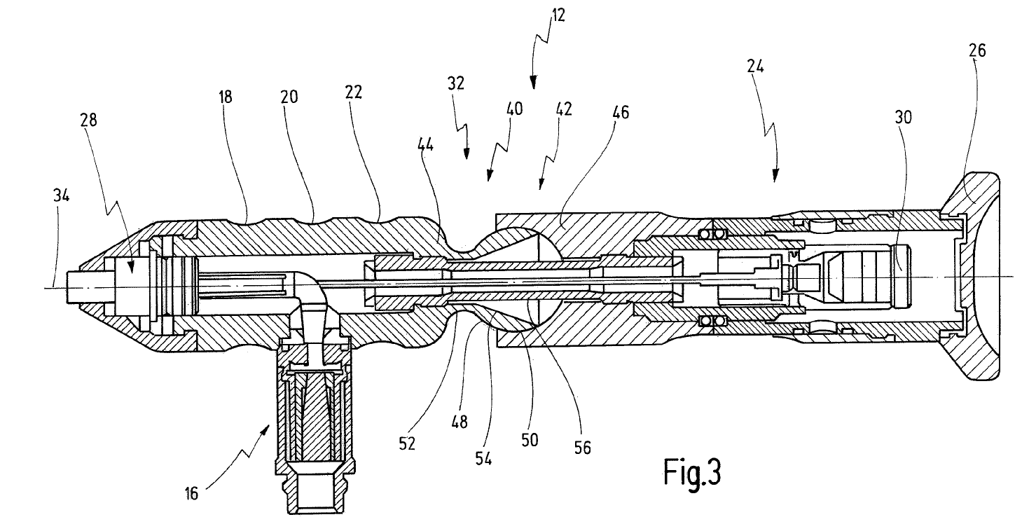

As represented in FIG. 3, an image carrying system 28 runs from the distal end of the endoscope shaft 14 to the eyepiece 30, which is arranged at the proximal end of the endoscope head 12.

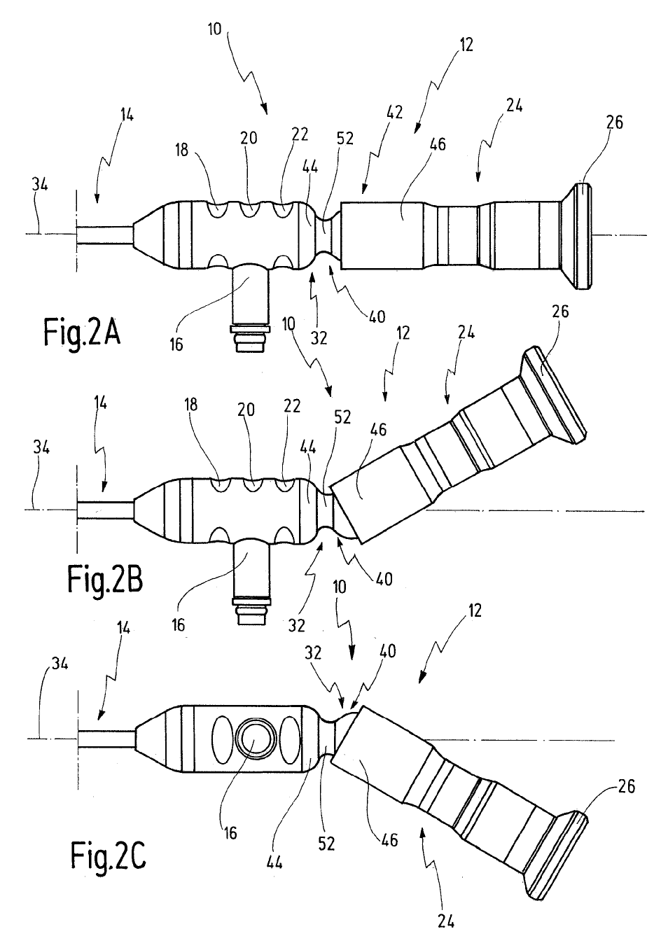

Arranged between the shaft 14 and the eyepiece 30 is a joint 32, with the result that the eyepiece 30 can be angled away in relation to the longitudinal direction 34 of the shaft 14. The longitudinal direction 34 of the shaft 14 designates the direction that is defined by the proximal end of the shaft 14. The joint 32, comprising at least two elements, is configured in such a way that the eyepiece 30 can be angled away in a first plane 36 in relation to the longitudinal direction 34 of the shaft 14. In addition, the joint 32 is configured such that it can be angled away in a second plane 38, transverse to the first plane 36. In FIGS. 2B and 2C, these planes 36, 38 correspond to the respective planes of the drawing. The maximum deflecting angle in a preferred configuration is in each case approximately 500 to both sides of the longitudinal direction 34 of the shaft 14.

In a first configuration, the joint 32 is formed as a ball joint 40 with a two-part housing 42 (see FIGS. 1 to 4). One housing part 44 is connected to the shaft 14, and a second housing part 46 is connected to the eyepiece 30. The first housing part 44 has a spherical outer surface 48, which is accommodated in a spherical inner surface 50 of the second housing part 46 in a sliding manner with frictional engagement. The frictional engagement offers the advantage that the second housing part is held with a self-locking effect in the first housing part 46 and maintains an angled-away position of the eyepiece 30 in relation to the longitudinal direction 34 of the shaft 14 even when the doctor is not grasping the second housing part 46. The spherical inner surface 50 of the second housing part 46 extends beyond the maximum outside diameter of the spherical outer surface 48 of the first housing part 44. This configuration has the advantage that the two housing parts 44, 46 hold each other without any further technical expenditure being required.

The first housing part 44 also has a radial constriction 52 on its side facing away from the second housing part 46. The radial constriction 52 tapers, seen in the longitudinal direction 34 of the shaft 14 away from the ball joint 40. The radial constriction 52 follows the spherical surface of the first housing part 44. The radial constriction 52 increases the deflecting angle of the second housing part 46 in relation to the longitudinal direction 34 of the shaft 14, since the second housing part 46 does not butt against the first housing part 44 over a considerable angular range. The ball joint 40 allows a deflection of the eyepiece 30 in relation to the longitudinal direction 34 of the shaft 14 in all planes that run through the centre point of the spherical proximal end of the first housing part 44. The maximum deflecting angle of the eyepiece 30 in relation to the longitudinal direction 34 of the shaft 14 is given by the two housing parts 44, 46 acting together as a stop, i.e., from a specific deflecting angle, the second housing part 46 butts against the first housing part 44. In this way it is prevented that the image carrying system 28 is interrupted in the region of the joint 32.

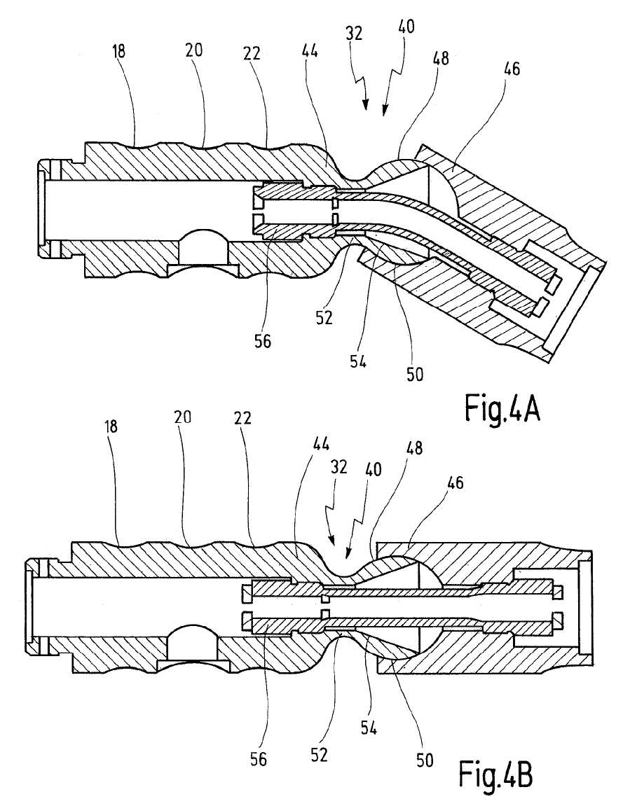

As represented in FIGS. 3, 4, the joint 32 also has a passage 54 for leading through a protective tube 56, in which the image carrying system 28 is accommodated. For the sake of maintaining an overview, the image carrying system 28 is not shown in FIG. 4. The passage 54 is configured in such a way that its surface is free from projections. As a result, damage to the image carrying system 28 by the inner side of the joint 32 is prevented. In addition, the protective tube 56 offers additional protection against the image carrying system 28 being damaged by the joint 32. The protective tube 56 is configured in such a way that it accommodates the image carrying system 28 over its full circumference and protects the image carrying system 28 from being damaged by the joint 32 from all sides equally.

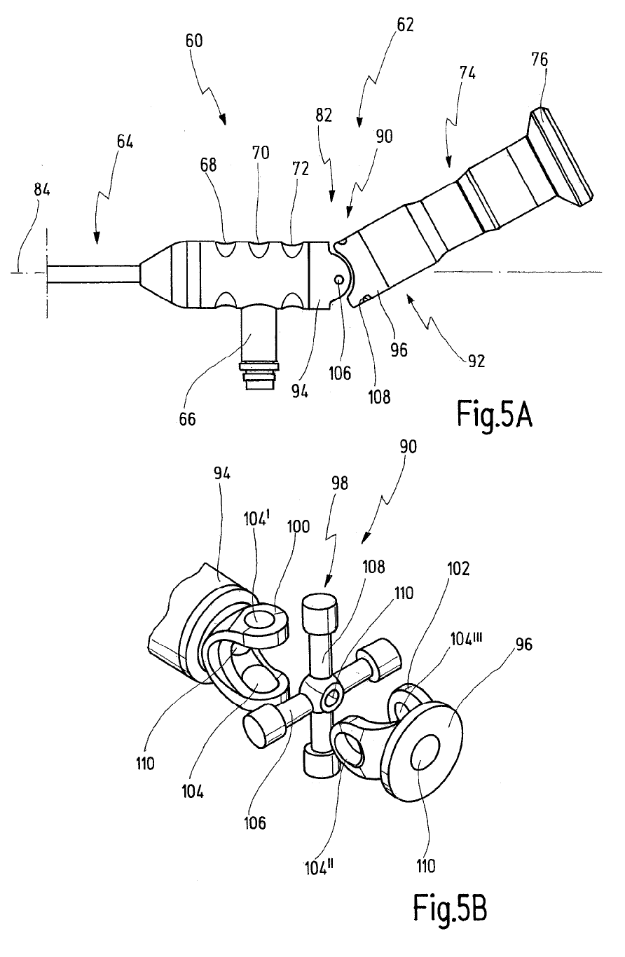

In FIGS. 5A and 5B, an endoscope 60 is represented, the features of the endoscope 60 that are the same as or correspond to those of the endoscope 10 being provided with reference numerals of the endoscope 10 incremented by 50.

An eyepiece 80 can be angled away in a first plane 86 in relation to a longitudinal direction 84 of a shaft 64 and in a second plane 88, which runs transversely in relation to the first plane 86. The angling away is permitted by a joint 82 formed as a cardanic joint 90.

The cardanic joint 90 has a two-part housing 92, a first housing part 94 being connected to the shaft 64 and a second housing part 94 being connected to the eyepiece 80. Arranged between the two housing parts 94, 96 are crossed axes 98. The two housing parts 94, 96 each have an arcuate element 100, 102, which are arranged transversely in relation to each other and engage in each other. Each arcuate element 100, 102 has two opposing openings 104-104′″. The crossed axes 98 have two fastening pins 106, 108, which run transversely in relation to each other and are connected to each other at their respective centre point. The ends of the fastening pins 106, 108 are accommodated in the openings 104-104′″ of the arcuate elements 100, 102. Like the ball joint 40, the cardanic joint 90 has a passage 110 through the joint 82. A protective tube 112 is led through the passage 110 of the cardanic joint 90. For the sake of maintaining an overview, the protective tube 112 is not represented in FIG. 5B.

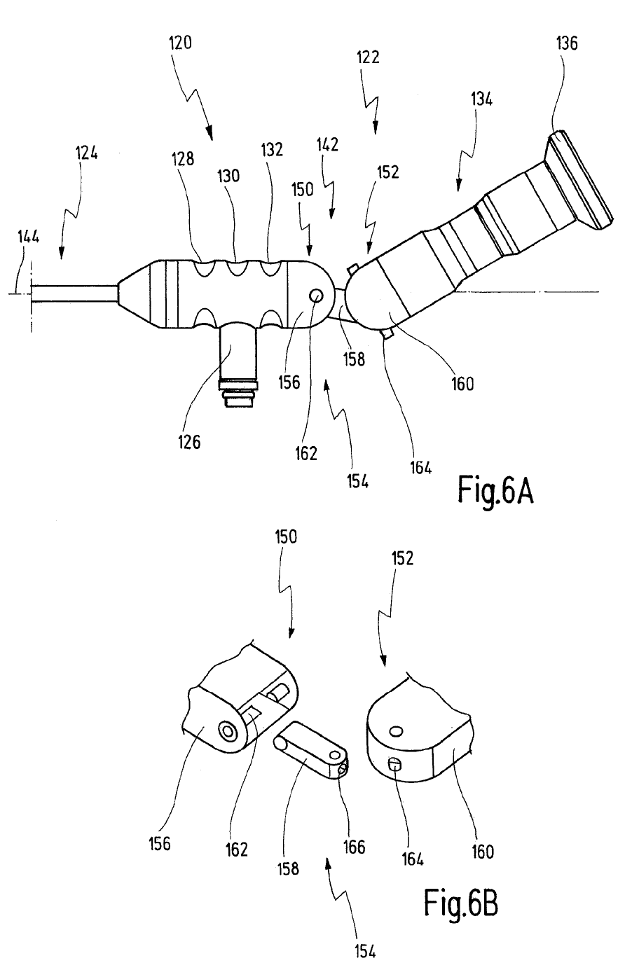

In FIGS. 6A and 6B, an endoscope 120 is represented, the features of the endoscope 120 that are the same as or correspond to those of the endoscope 10 being provided with reference numerals of the endoscope 10 incremented by 110.

An eyepiece 140 can be angled away in a first plane 146 in relation to a longitudinal direction 144 of a shaft 124 and in a second plane 148, which runs transversely in relation to the first plane 146. The angling away is permitted by a joint 142 that is formed as at least two spaced-apart 1-axis joints 150, 152.

In the representation shown, the joint 142 has a three-part housing 154, a first housing part 156 being connected to the shaft 124 and via the 1-axis joint 150 to a second housing part 158. The second housing part 158 is connected via the second 1-axis joint 152 to a third housing part 160. The third housing part 160 is connected to an eyepiece 140. Each 1-axis joint 150, 152 has a pin 162, 164, which connects the adjoining housing parts 156, 158, 160 to one another in a jointed manner. Since the two pins 162, 164 are arranged transversely in relation to each other, the first 1-axis joint 150 can be angled away in a plane 146 in relation to a longitudinal direction 144 of the shaft 124 and in a second plane 146 that is transverse to the first plane 144. In the representation shown of the two 1-axis joints 150, 152, the pins 162, 164 are interrupted in such a way that a passage 166 for accommodating a light guiding system 138 runs through the joint. A protective tube 168 is led through the passage 166 of the joint 142. For the sake of maintaining an overview, the protective tube 168 is not represented in FIG. 6B.

What is claimed is:

1. An endoscope for the intubation of a respiratory tract, comprising
a shaft defining a longitudinal direction and having a proximal end,
an endoscope head arranged at said proximal end of said shaft and carrying an eyepiece,
a joint arranged on said endoscope head between said shaft and said eyepiece for angling said eyepiece away in relation to said longitudinal direction of said shaft, said joint being formed in such a way that said eyepiece can be angled away in a first plane and at least in a second plane which runs transversely in relation to said first plane.

2. The endoscope of claim 1, wherein said joint is formed in such a way that said eyepiece can be angled away, at least in one of said first or second planes, up to an angle of approximately 50° in relation to said longitudinal direction of said shaft.

3. The endoscope of claim 1, wherein said joint is formed in such a way that said eyepiece can be angled away, at least in one of said first or second planes, up to an angle of approximately 40° in relation to said longitudinal direction of said shaft.

4. The endoscope of claim 1, wherein said joint is formed in such a way that said eyepiece can be angled away, at least in one of said first or second planes, up to an angle of approximately 30° in relation to said longitudinal direction of said shaft.

5. The endoscope of claim 1, wherein said joint is formed as a ball joint.

6. The endoscope of claim 5, wherein said ball joint has a two-part housing, a first housing part being connected to said shaft and a second housing part being connected to said eyepiece.

7. The endoscope of claim 6, wherein said first housing part is accommodated in said second housing part in a sliding manner with frictional engagement.

8. The endoscope of claim 7, wherein said first housing part has a spherical outer surface and said second housing part has a spherical inner surface, in which said first housing part is accommodated.

9. The endoscope of claim 8, wherein said first housing part is arranged in said second housing part in such a way that said second housing part extends beyond a maximum outside diameter of said spherical outer surface of said first housing part.

10. The endoscope of claim 9, wherein said spherical outer surface of said first housing part has a radial constriction on a side facing away from said second housing part.

11. The endoscope of claim 10, wherein said radial constriction tapers, seen in said longitudinal direction of said shaft, away from said joint.

12. The endoscope of claim 6, wherein said two housing parts act together as a stop to limit the angling away of said eyepiece.

13. The endoscope of claim 1, wherein said joint is formed as a cardanic joint.

14. The endoscope of claim 13, wherein said cardanic joint has a two-part housing and two crossed axes, a first housing part being connected to said shaft and a second housing part being connected to said eyepiece, and said crossed axes being arranged between said two housing parts.

15. The endoscope of claim 1, wherein said joint has at least two spaced-apart 1-axis joints.

16. The endoscope of claim 1, wherein said joint has a passage for leading through an image carrying system, said passage being free from projections.

17. The endoscope of claim 16, wherein said joint has a protective tube for said image carrying system, said protective tube extending through said passage of said joint and accommodating said image carrying system.

18. The endoscope of claim 17, wherein said protective tube extends around said image carrying system over a full circumference of said image carrying system.
